(19) United States
(12) Patent Application Publication
(10) Pub. No.: US 2018/0021285 A1
(43) Pub. Date: Jan. 25, 2018

(54) METHOD FOR VISUALIZATION OF CALCIUM CONTAINING COMPONENTS IN BIOLOGICAL SYSTEMS

(71) Applicant: Visikol Inc., New Burnswick, NJ (US)

(72) Inventors: Nicholas Ingram Crider, Franklin Park, NJ (US); Thomas Steven Villani, JR., Franklin Park, NJ (US); Michael Thomas Johnson, Somerville, NJ (US)

(21) Appl. No.: 15/659,372

(22) Filed: Jul. 25, 2017

Related U.S. Application Data
(60) Provisional application No. 62/366,238, filed on Jul. 25, 2016.

(51) Int. Cl. A61K 31/221; C09B 1/02; C07C 229/24; C07C 229/26; C07F 3/04

(52) U.S. Cl. CPC A61K 31/221; C07C 229/26; C07F 3/04; C07C 229/24; C09B 1/02

(57) ABSTRACT

Provided are compositions and methods for visualizing the skeletal tissues of a biological specimen, comprising treating the specimen with a calcium chelating agent, a staining solution, and a clearing solution.

### CROSS- REFERENCE TO RELATED APPLICATIONS

This application claims the benefit of U.S. Provisional Application No. 62/366,238, filed Jul. 25, 2016. The entire teachings of the above application are incorporated herein by reference.

### FIELD OF THE INVENTION

The present application relates generally to anatomy, and more specifically to the visualization of calcium-containing components in biological systems.

### BACKGROUND

Bone is an important organ for mineral homeostasis and metabolism. Chemical staining of calcium-containing tissues, including bones, cartilage and other components of the skeletal system, is the most direct and reliable strategy to view it in situ, whether it be in an embryo or adult animal. Also, skeletal visualization provides a convenient way of evaluating the late embryonic and fetal skeleton, which is an integral step in many mammalian teratological studies.

To visualize calcium-containing tissue in animals, stains have been used after digestion of non-calcified peripheral tissue. For example, potassium hydroxide is used to digest the specimen, and then Alizarin Red S is applied for staining (Dawson (1926) Stain Technol. 1:123-124). An automated technique for double-staining has also been developed which can differentiate bone from cartilage (Trueman et al. (1998) Biotech. Histochem. 74:98-104).

In these conventional methods, a base solution is often used to digest/macerate the peripheral tissues where the calcium dyes will also bind non-specifically, and thus improve the visibility of the skeletal system. However, when removing the non-specifically bound calcium dye, care must be taken not to over-digest the specimen, which leads to dissolution of connective tissue and subsequent destruction of the skeletal system. In addition, variations in temperature, ionic strength, ion concentration, and pH can have major effects on the outcome of the procedure, so its management is necessary for a positive result. In light of these drawbacks, toxicologists have long sought alternative non-destructive methods for skeletal visualization.

Magnetic resonance imaging (MRI, Schmidt et al. (2007) Skeletal Radiol. 36:1109-1119), computed tomography (Hiller et al. (2015) Isr. Med. Assoc. J. 17:42-6), and X-ray (Ritz et al. (1978) Kidney Int. 13:316-23) are some examples of the non-destructive methods that have been used to image the skeletal systems of vertebrates and invertebrates. In addition, high-resolution microcomputed tomography has also been used (Chityala et al. (2013) J. Digit. Imaging 26:302-308). However, the major drawback to the known non-destructive methods is the high costs associated with the acquisition and operation of the radiological equipment.

Thus, what is needed is an improved method of visualizing calcium-containing components of a specimen, such as the skeletal system, in a non-destructive way without any specialized and expensive equipment, which leaves soft tissue intact, while reducing non-specific staining of tissue other than bone and facilitating the localization of the staining dyes.

### SUMMARY

It has been discovered that pretreating a tissue specimen with a calcium chelating agent such as ethylenediaminetetraacetic acid (EDTA) facilitates visualization of the calcium-containing components within the tissue. This discovery has been exploited to provide the present disclosure, which, in part, includes a method of visualizing skeletal components in a tissue specimen.

In one aspect, the method comprises contacting a specimen with a calcium chelating agent. The specimen is then stained with a dye, and then contacted with a clearing agent to cause the tissue to become transparent. In some embodiments, the specimen is washed before it is stained, and in some embodiments, the stained specimen is washed before it is contacted with the clearing agent.

In some embodiments, the specimen is an embryo, a juvenile or adult vertebrates, such as a mammal, fish, reptile, amphibian, or bird. In other embodiments, the specimen is an invertebrate, fungus, bacteria, or plant.

In certain embodiments, the calcium chelating agents are EDTA, diethylenetriamine pentaacetic acid (DTPA), hydroxyethylethylenediaminetriacetic acid (HEDTA), nitrilotriacetic acid (NTA), ethylene glycol tetraacetic acid (EGTA), or 1,2-bis(o-aminophenoxy)ethane-N,N,N′,N′-tetraacetic acid (BAPTA) and/or combinations thereof. In a specific embodiment, EDTA is used for this purpose. In certain embodiments, the EDTA is present at a concentration of 0.1 to 100 mM, 1 mM to 100 mM, 20 mM to 80 mM, 1 mM to 100 mM, 15 mM to 80 mM, 10 mM to 90 mM, 20 mM to 75 mM, 30 mM 60 mM, 40 mM to 50 mM, about 10 mM, about 20 mM, about 30 mM, about 40 mM, about 50 mM, about 60 mM, about 70 mM, about 80 mM, about 90 mM, or about 100 mM.

In some embodiments, staining is done with Alizarin red, Alizarin red S, Alizarin Yellow, Alizarin Yellow GG, Alizarin-3-methylimidoacetic acid, alizarin blue black B, and other derivatives of Alizarin that chelate/interact/bond to Ca⁺² ions; purpurin, von Kossa's phthalocyanin, and glyoxal bis(2-hydroxyanil), murexide, 8-hydroxyquinoline, Alcian blue 8GX and other derivatives, or a combination thereof. In a particular embodiment, both Alizarin red S and Alcian blue 8GX are used.

In some embodiments, the clearing agent is glycerol, water, and/or an aqueous surfactant mixture (e.g. 0.1%-1%). In certain embodiments the aqueous surfactant mixture comprises polyoxyethylene octyl phenyl ether in water, sodium dodecyl sulfate in water) (about 0.001%-50%, about 0.01% to about 5%, about 5% to about 25%, about 25% to about 50%, or about 1%).

In other embodiments, the clearing solution comprises glycerol (15% to 70%, 20% to 65%, 25% to 60%, 30% to 55%, 35% to 50%, 40% to 40%, about 15%, about 20%, about 25%, about 30%, about 35%, about 40%, about 45%, about 50%, about 55%, about 60%, about 65%, about 70%, water, Visikol Clearing Solutions), is urea (about 10 mM to about 4 M, about 50 mM to about 2 M, or about 1 M), water/ethanol (about 0.1%-100%, or about 30% aqueous solution), isopropanol (about 0.1%-100%, about 5% to about 90%, about 20% to about 80%, about 25% to about 70%, or about 30%), n-butanol (100%), NaOH (about 10 mM to 1 M, about 20 mM to 80 mM, about 30 mM to about 70 mM, about 40 mM to about 60 mM, or about 50 mM), LiOH (about 10 mM to 1 M, about 20 mM to 80 mM, about 30 mM to about 70 mM, about 40 mM to about 60 mM, or about 50 mM), Ca(OH)₂ (about 10 mM to 1 M, about 20 mM to 80 mM, about 30 mM to about 70 mM, about 40 mM to about 60 mM, or about 50 mM), and other hydroxide base solutions (about 10 mM to 1 M, about 20 mM to 80 mM, about 30 mM to about 70 mM, about 40 mM to about 60 mM, or about 50 mM), xylene (100%), xylitol (100%), d-limonene (100%), FocusClear® (Cedarlane Corporation, Burlington, N.C.), HistoClear® (National Diagnostics, Atlanta, Ga.), benzene (100%), toluene (100%), benzyl alcohol (100%), benzyl benzoate (neat), benzyl ether (neat), methyl salicylate (neat), mono or dichloro-salicylate (neat), thiodiethanol (neat), formamide (neat), and fructose solutions (about 100 mg/g to about 4 g/g about 10 mg/g to about 3 g/g, about 500 mg/g to about 3 g/g, about 1 g/g to about 2 g/g, or about 1 g/g), glycerol (about 0.1% to about 100%, about 0.1% to about 99.9%, about 0.5% to about 95%, about 1% to about 90%, about 15% to about 80%, about 20% to about 70%, about 30% to about 50%, about 40% to about 50%, or about 30%), chloral hydrate (about 1% to about 10%), alcoholic, alcohol glycerol (about 0.1% to about 99%, about 0.5% to about 90%, about 1% to about 80%, about 15% to about 70%, about 20% to about 60%, or about 50%), alcoholic solutions, alcohols include methanol, ethanol, isopropanol, or ethanol (at about 50%), Visikol® (any grade or formula, Visikol Inc. New Brunswick, N.J.). In one embodiment, Visikol® solution is used as the clearing agent.

The disclosure further provides a kit for visualizing a calcified component in a tissue specimen, the kit comprising a chelating agent, calcium binding dye, and a clearing solution.

In some embodiments, the kit further includes distilled water (d H₂O), a KOH solution, a bacteriostatic, a surfactant, instructions, or combinations thereof. In certain embodiments, the bacteriostatic comprises azide.

In another aspect the disclosure provides a kit for a visualizing a calcified component in a tissue specimen. The kit comprises a chelating agent, a calcium-binding dye, and a clearing solution.

In some embodiments the chelating agent is, EDTA, diethylenetriamine pentaacetic acid (DTPA), hydroxyethylethylenediaminetriacetic acid (HEDTA), nitrilotriacetic acid (NTA), ethylene glycol tetraacetic acid (EGTA), or 1,2-bis(o-aminophenoxy)ethane-N,N,N′,N′-tetraacetic acid (BAPTA) and/or combinations thereof. In a specific embodiment, EDTA is used for this purpose. In certain embodiments, the EDTA is present at a concentration of 0.1 to 100 mM, 1 mM to 100 mM, 20 mM to 80 mM, 1 mM to 100 mM, 15 mM to 80 mM, 10 mM to 90 mM, 20 mM to 75 mM, 30 mM 60 mM, 40 mM to 50 mM, about 10 mM, about 20 mM, about 30 mM, about 40 mM, about 50 mM, about 60 mM, about 70 mM, about 80 mM, about 90 mM, or about 100 mM.

In Certain embodiments, the dye comprises Alizarin red, Alizarin red S, Alizarin Yellow, Alizarin Yellow GG, Alizarin-3-methylimidoacetic acid, alizarin blue black B, and other derivatives of Alizarin that chelate/interact/bond to Ca⁺² ions; purpurin, von Kossa's phthalocyanin, and glyoxal bis(2-hydroxyanil), murexide, 8-hydroxyquinoline, Alcian blue 8GX and other derivatives, or a combination thereof. In a particular embodiment, both Alizarin red S and Alcian blue 8GX are used.

In some embodiments, the clearing solution comprises glycerol, water and/or an aqueous surfactant mixture. In certain embodiments, the clearing solution comprises glycerol (15% to 70%, 20% to 65%, 25% to 60%, 30% to 55%, 35% to 50%, 40% to 40%, about 15%, about 20%, about 25%, about 30%, about 35%, about 40%, about 45%, about 50%, about 55%, about 60%, about 65%, about 70%, water, Visikol Clearing Solutions), is urea (about 10 mM to about 4 M, about 50 mM to about 2 M, or about 1 M), water/ethanol (about 0.1%-100%, or about 30% aqueous solution), isopropanol (about 0.1%-100%, about 5% to about 90%, about 20% to about 80%, about 25% to about 70%, or about 30%), n-butanol (100%), NaOH (about 10 mM to 1 M, about 20 mM to 80 mM, about 30 mM to about 70 mM, about 40 mM to about 60 mM, or about 50 mM), LiOH (about 10 mM to 1 M, about 20 mM to 80 mM, about 30 mM to about 70 mM, about 40 mM to about 60 mM, or about 50 mM), Ca(OH)₂ (about 10 mM to 1 M, about 20 mM to 80 mM, about 30 mM to about 70 mM, about 40 mM to about 60 mM, or about 50 mM), and other hydroxide base solutions (about 10 mM to 1 M, about 20 mM to 80 mM, about 30 mM to about 70 mM, about 40 mM to about 60 mM, or about 50 mM), xylene (100%), xylitol (100%), d-limonene (100%), FocusClear® (Cedarlane Corporation, Burlington, N.C.), HistoClear® (National Diagnostics, Atlanta, Ga.), benzene (100%), toluene (100%), benzyl alcohol (100%), benzyl benzoate (neat), benzyl ether (neat), methyl salicylate (neat), mono or dichloro-salicylate (neat), thiodiethanol (neat), formamide (neat), and fructose solutions (about 100 mg/g to about 4 g/g about 10 mg/g to about 3 g/g, about 500 mg/g to about 3 g/g, about 1 g/g to about 2 g/g, or about 1 g/g), glycerol (about 0.1% to about 100%, about 0.1% to about 99.9%, about 0.5% to about 95%, about 1% to about 90%, about 15% to about 80%, about 20% to about 70%, about 30% to about 50%, about 40% to about 50%, or about 30%), chloral hydrate (about 1% to about 10%), alcoholic, alcohol glycerol (about 0.1% to about 99%, about 0.5% to about 90%, about 1% to about 80%, about 15% to about 70%, about 20% to about 60%, or about 50%), alcoholic solutions, alcohols include methanol, ethanol, isopropanol, or ethanol (at about 50%), Visikol® (any grade or formula, Visikol Inc. New Brunswick, N.J.). In one embodiment, Visikol® solution is used as the clearing agent.

In some embodiments, the kit further comprises KOH, a bacteriostatic, and/or instructions for use. In some embodiments, the bacteriostatic is azide.

### BRIEF DESCRIPTION OF THE FIGURES

The foregoing and other objects of the present disclosure, the various features thereof, as well as the invention itself may be more fully understood from the following description, when read together with the accompanying drawings in which:

FIG. 1 is diagrammatic representations of an embodiment of the method for skeletal visualization;

FIG. 2 is a photographic representation of a fresh fetal mouse after skeletal visualization using the instant method;

FIG. 3A is a photographic representation of a dehydrated GD21 rat fetus stored for 24 hours in 100% isopropanol;

FIG. 3B is a photographic representation of a previously-frozen GD21 rat fetus;

FIG. 4A is a photographic representation of the dehydrated GD21 rat fetus pretreated with 100 mM EDTA for 2 hours;

FIG. 4B is a photographic representation of the previously-frozen GD21 rat fetus pretreated with 100 mM EDTA for 2 hours;

FIG. 5A is a photographic representation of the dehydrated GD21 rat fetus treated for 24 hours with 0.003% Alizarin Red S, 1% KOH;

FIG. 5B is a photographic representation of the previously-frozen GD21 rat fetus treated for 24 hours with 0.003% Alizarin Red S, 1% KOH;

FIG. 6A is a photographic representation of the dehydrated GD21 rat fetus treated with deionized (DI) water for 6 hours;

FIG. 6B is a photographic representation of the previously-frozen GD21 rat fetus treated with DI water for 6 hours;

FIG. 7A is a photographic representation of the dehydrated GD21 rat fetus cleared with 30% glycerol, 0.1% Triton X-100 in DI water overnight;

FIG. 7B is a photographic representation of the previously-frozen GD21 rat fetus cleared with 30% glycerol, 0.1% Triton X-100 in DI water overnight; and

FIG. 8 is a photographic representation of GD28 a fetal rabbit after skeletal visualization using the instant method.

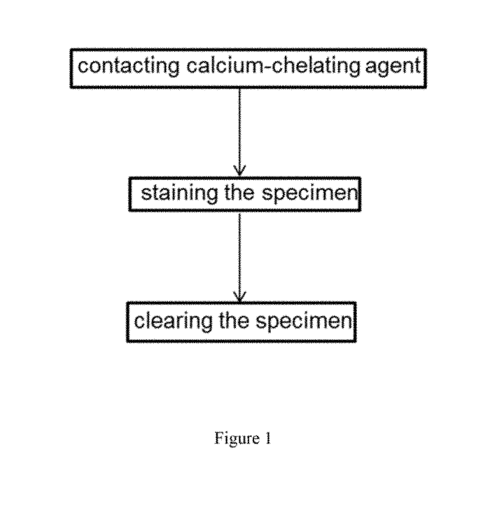

### DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

Unless otherwise defined, all technical and scientific terms used herein have the same meaning as commonly understood by one of ordinary skill in the art to which this disclosure belongs.

The term “dehydrated” refers to a state wherein there is no water present. The term “chelating agent” refers to a molecule having two or more unshared electron pairs available for donation to a metal ion. Typically, the electron pairs of a chelating agent forms coordinate bonds with a single metal ion; however, in certain examples, a chelating agent may form coordinate bonds with more than one metal ion, with a variety of binding modes being possible. Alizarin Red S may chelate or bind with calcium in different forms such as:

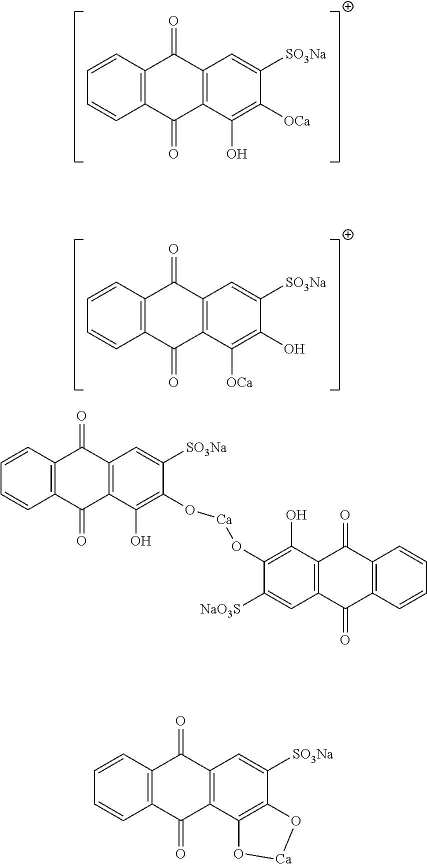

and the like, wherein calcium may chelate with either phenol group in the Alizarin structure, or both phenol groups in the Alizarin structure. When calcium chelates with multiple phenol groups, the chelation may be formed intramolecularly or intermolecularly.

The present disclosure is a method of visualizing the calcium-containing components of living specimens, including plants, vertebrates and/or portions thereof. Plants of interest include calcium-containing plants such as Arabidopsis thaliana, Origanum vulgaris, Ocimum basillicum. Vertebrates whose skeletal systems can be visualized include, but are not limited to fish, reptiles, amphibians, mammals, and birds. The vertebrate sample can be fetal, juvenile, and/or adult, or a portion thereof such as a limb. The specimen may be fresh, frozen, dehydrated, or fixed with a precipitative (e.g., ethanol) and/or cross-linking fixative (e.g., formaldehyde) before processing.

If alive, the animal is euthanized, e.g., with carbon dioxide, nembutol, or other approved method, and eviscerated to remove organs so the view of skeleton is not obstructed. Typically as a necropsy procedure, the abdominal cavity is opened and all internal viscera are removed; ribcage can be sliced through cartilage or left intact. The skin is slit around the back of the neck and the fat pads surrounding the vertebrate can be removed or left intact.

If the specimen is not being examined at that time, it may be frozen. For example, the specimen is placed in an environment of a temperature which is gradually lowered to freeze so the specimen is not shattered, using, e.g., a lab freezer, down to about −5° C. to about −80° C.

Alternatively, if the specimen is not being examined at that time, fresh specimen may also be fixed using solvents, including but not limited to methanol (>the 30% mixture with water), ethanol (>the 30% mixture with water), isopropanol (>the 30% mixture with water), n-propanol (100%), acetic acid (0.1% -20% in water, methanol, or ethanol), and combinations thereof, acetone (100%), AMEX fixation (acetone, methylbenzoate, and xylene in series, see Sato et al. Amer. J. Pathol. (1992) 140(4):775-779), methyl ethyl ketone (neat), aqueous glutaraldehyde mixtures (1% -99% or 100%), and HOPE® fixative (Polysciences, Inc. Warrington, Pa.). Fixation can be done prior to or at the time of freezing. The specimen is thawed before using, this step should not take long in order to avoid specimen decay. The methods used to thaw the specimen include, but are not limited to placing in a 0-5° C. lab refrigerator overnight, leaving at room temperature (RT) (about 18 to about 25° C.) for about 3 hours, or placing in warm water (about 25 to about 85° C.) for approximately 20 minutes.

The specimen is then submerged in a calcium chelating agent. To remove most of the calcium-containing components out of the soft tissue, while leaving the bone components intact. Some non-limiting examples of useful, commercially-available calcium chelating agents include: EDTA, diethylenetriaminepentaacetic acid (DTPA), hydroxyethylethylenediaminetriacetic acid (HEDTA), nitrilotriacetic acid (NTA), ethylene glycol tetraacetic acid (EGTA), 1,2-bis(o-aminophenoxy)ethane-N,N,N′,N′-tetraacetic acid (BAPTA), deferoxamine mesylate, N,N,N′,N′-tetrakis-(2-pyridylmethyl)ethylenediamine, ethylene-bis(oxyethylenenitrilo)tetraacetic acid, 6-bromo-N′-(2-hydroxybenzylidene)-2-methylquinoline-4-carbohydrazide, Fura 2, Fura 2-acetoxymethyl ester (Fura-2AM), indo-1, fluo-3, fluo-4, Calcium Green-1,2,2′-bipyridine, 1,3-propylenediaminetetraacetic acid, or any of the other chelators referenced by Durham (Cell Calcium (1983) 4:33-46); Pethig et al. (Cell Calcium (1989) 10:491-498). Additionally, calcium-binding dyes such as those referenced by Barth et al. (Photochem. Photobio. Sci. (2006) 5:107-115) may be used if the coloration produced by the dye does not preclude visualization of the skeleton. Combinations or analogs of these agents may also be used.

One non-limiting, representative calcium chelating agent is EDTA. Some non-limiting effective concentrations of EDTA are from about 1 mM to about 1,000 mM, from about 10 mM to about 800 mM, from about 20 mM to about 500 mM, from about 1 mM to about 500 mM, from about 20 mM to about 1,000 mM, or about 50 mM, or about 80 mM, about 100 mM. Urea can be added to facilitate penetration of the chelating reagent and to account for tap water hardness. Some non-limiting effective urea concentrations are from about 0.01 μM to about 4 M, from about 10 mM to about 2 M, from about 1 M to about 4 M, from about 0.1 μM to about 1 M, from about 1 μM to about 6 M, from about 0.01 μM to about 1 mM, from about 4 M to about 6 M, or about 4 M, about 2 M, or about 1 M.

This calcium chelating solution has pH values from about 5 to about 12. The type, size, and age of the specimen may dictate the incubation time, temperature, and volume of the calcium chelating solution used. However, it is within the skilled artisan's purview to determine the optimal conditions to obtain a good visualization effect within an acceptable time limit. For example, larger amounts of calcium chelating solution are typically used for larger and thicker specimens. The time allocated for pre-treatment also depends on the specimen type and size, with smaller specimens requiring less time and larger and whole specimens requiring more time. For example, some effective pre-treatment times are from about 1 min to about 96 hours, from about 5 minutes to about 72 hours, from about 10 minutes to about 48 hours, from about 30 minutes to about 36 hours, or from about 1 hour to about 24 hours. Some non-limiting effective serving temperatures are about RT for manual processing, and about 40° C. for automated processing (e.g. using the Sakura TissueTek® VIP, Sakura Finetek USA, Inc. Torrance, Calif., or similar tissue processing instrument).

In the next step, the treated specimen is contacted with a staining agent specific for calcium-containing components. Some non-limiting examples of useful, commercial-available staining agents include Alizarin and its derivatives, Alcian and its derivatives, Calcein and its derivatives, xylenol orange, Oregon green and related derivatives, von Kossa, Oil Red O, Goldner's trichrome, haematoxylin and eosin stain, and any other stain or fluorescent probe which is selective for calcium or phosphate (see, Hamilton et al., Chem. Soc. Rev. (2015) 44:4415-4432).

Some non-limiting effective concentrations of Alizarin Red S stain solution are from about 0.0000001% to about 0.005%, from about 0.005% to about 1%, from about 0.001% to about 0.5%, from about 0.0005% to about 0.05%, from about 0.000005% to about 0.1%, from about 0.00001% to about 0.005%, from about 0.001% to about 0.1%, from about 0.001% to about 0.5%, or about 0.00005%, about 0.0001%, about 0.0005% or about 0.001%, or about 0.005%. The concentration is either in weight or volume percentage.

A basifying compound may be added into the Alizarin Red S solution in order to adjust pH and increase binding of stain to calcium constituents of tissue. Some non-limiting effective concentrations of a typical base, KOH, are from about 0.0001% to about 1%, from about 1% to about 4%, from about 0.001% to about 0.5%, from about 0.005% to about 0.5%, from about 1% to about 2%, from about 0.0001% to about 0.5%, from about 0.01% to about 5%, or about 0.05%, about 0.1%, about 0.5% or about 1%, or about 2%. The concentration is either in weight or volume percentage. Additives and/or chelators such as, but not limited to, EDTA can also be added to modulate calcium content of the water. Some non-limiting effective concentrations of EDTA are from about from about 1 mM to about 1,000 mM, from about 10 mM to about 800 mM, from about 20 mM to about 500 mM, from about 1 mM to about 500 mM, from about 20 mM to about 1,000 mM, or about 50 mM, or about 80 mM, about 100 mM. As with the calcium chelating solution, the amount of staining solution and the time required for staining depend on the specimen type and size. Staining may be done at about RT (18-25° C.) for manual processing, and about 10° C. to about 70° C. for automated processing.

In some cases, a double-staining method may be used. In this method, the specimen is submerged in an alcohol, for example 100% isopropanol, for about 1 hour to 12 hours. The specimen is then removed from the alcohol and placed into a solution containing 25% (v/v) acetic acid, 0.025% (w/v) Alcian 8GX, dissolved in ethanol (or any other collagen or cartilage specific stain referenced in Handbook of Histology Methods for Bone and Cartilage (An & Martin, Springer 2006), for about 1 hour to 24 hours. The specimen is then removed from the stain solution and transferred to an alcohol, for example 100% isopropanol, for about 1 hour to 24 hours, in order to remove excess, non-adhered staining solution. The specimen is then processed as described in paragraph 33.

A washing agent may be used to remove excess, non-adhered staining solution wash the specimen after staining before the clearing step is carried out. For example, post-stain washing is useful for vertebrate specimens with skins. Some non-limiting examples of useful post-stain washing agents include: decalcified, demineralized, deionized, and/or (singly, doubly or further) distilled water. Post-stain washing is useful for vertebrate specimens with skins. Urea can be added into the washing solution, with a concentration from about 1 μM to about 4 M. The specimen is submerged in the washing solution for about 1 minute to about 48 hours before it is removed. This washing step can be repeated as many times as desired, and is performed at about RT for manual processing, and about 10° C. to about 70° C. for automated processing.

In another step, the stained specimen is submerged in a clearing agent to clarify the tissue around the stained calcium-containing components. Some non-limiting examples of useful clearing agents include water (tap, decalcified, demineralized, deionized, and/or singly, doubly or further distilled water), aqueous salt solutions (10 mM to 4M) including but not limited to phosphates, sulfates, chlorides, metal ions, the clearing solution is, urea (about 10 mM to about 4 M, about 50 mM to about 2 M, or about 1 M), water/ethanol (about 0.1%-100%, or about 30% aqueous solution), isopropanol (about 0.1%-100%, about 5% to about 90%, about 20% to about 80%, about 25% to about 70%, or about 30%), n-butanol (100%), NaOH (about 10 mM to 1 M, about 20 mM to 80 mM, about 30 mM to about 70 mM, about 40 mM to about 60 mM, or about 50 mM), LiOH (about 10 mM to 1 M, about 20 mM to 80 mM, about 30 mM to about 70 mM, about 40 mM to about 60 mM, or about 50 mM), Ca(OH)₂ (about 10 mM to 1 M, about 20 mM to 80 mM, about 30 mM to about 70 mM, about 40 mM to about 60 mM, or about 50 mM), and other hydroxide base solutions (about 10 mM to 1 M, about 20 mM to 80 mM, about 30 mM to about 70 mM, about 40 mM to about 60 mM, or about 50 mM), xylene (100%), xylitol (100%), d-limonene (100%), FocusClear® (Cedarlane Corporation, Burlington, N.C.), HistoClear (National Diagnostics, Atlanta, Ga.), benzene (100%), toluene (100%), benzyl alcohol (100%), benzyl benzoate (neat), benzyl ether (neat), methyl salicylate (neat), mono or dichloro-salicylate (neat), thiodiethanol (neat), formamide (neat), and fructose solutions (about 100 mg/g to about 4 g/g about 10 mg/g to about 3 g/g, about 500 mg/g to about 3 g/g, about 1 g/g to about 2 g/g, or about 1 g/g), glycerol (about 0.1% to about 100%, about 0.1% to about 99.9%, about 0.5% to about 95%, about 1% to about 90%, about 15% to about 80%, about 20% to about 70%, about 30% to about 50%, about 40% to about 50%, or about 30%), chloral hydrate (about 1% to about 10%), alcoholic, alcohol glycerol (about 0.1% to about 99%, about 0.5% to about 90%, about 1% to about 80%, about 15% to about 70%, about 20% to about 60%, or about 50%), alcoholic solutions, alcohols include methanol, ethanol, isopropanol, or ethanol (at about 50%), Visikol® (any grade or formula). In one embodiment, Visikol® solution is used as the clearing agent. Also, clearing agents and methodologies referenced by Tainaka et al. (Annu. Rev. Cell Dev. Biol. (2016), 32:9.1-9.29) are also useful.

One non-limiting, representative clearing agent is Visikol, commercially available from Visikol Inc. (WO 2013/155064). Again, the amount of clearing solution to be used depends on the specimen type and size. The specimen is submerged in the clearing solution, for a time dependent on the specimen type and size. The clearing step is done at about RT for manual processing, and about 10° C. to about 70° C. for automated processing.

For example, where a fetal mouse is the specimen, it can be placed in about 20 mL calcium chelating solution (25 mM EDTA, 1 M urea) for about 0.5 hour to about 1 hour, stained in about 0.005% Alizarin red S, 1% KOH for about 23 hours, washed with about 10 mL to about 20 mL water for about 4 hours, and cleared for about 2 hours with about 10 mL to about 20 mL clearing solution.

In other examples where a fetal rat is used as the specimen, it is submerged in about 30 mL 50 mM EDTA, 1 M urea, pH 8, incubated for about 1 hour to about 1.5 hours, and then stained with 0.005% Alizarin red S, 1% KOH for about 23 hours, washed with deionized (DI) water (50 mL) for about 4 hours, and then cleared with about 20 mL to about 50 mL clearing solution for about 4 hours.

In another example, when a fetal rabbit specimen is used, it is submerged in about 50 mL to about 100 mL 100 mM EDTA, 1 M urea, pH 8, incubated for about 2 hours, and then stained with 0.005% Alizarin red S, 1% KOH for about 23 hours. It is washed with about 50 mL to about 100 mL DI water for about 4 hours, and cleared with about 50 mL to about 100 mL clearing solution for about 4 hours.

After treatment, the specimen may be stored submerged in clearing solution or transferred to a preservation or storage solution such as, but not limited to urea (10 mM to 4M aqueous solution, or about 1M), water/ethanol (about 0.1% to about 100% aqueous mixture, or 30% aqueous solution), isopropanol (about 0.1%-about 100% aqueous mixture, or 30% aqueous solution), n-butanol (100%), NaOH (about 10 mM to about 1M, or 50 mM), LiOH (about 10 mM to about 1M, or 50 mM), Ca(OH)₂ (about 10 mM to about 1M, or 50 mM) and other hydroxide base solutions (about 10 mM to about 1M, or 50 mM), xylene (100%), xylitol (100%), d-limonene (100%), FocusClear® (Cedarlane Corporation, Burlington, N.C.), HistoClear® (National Diagnostics, Atlanta, Ga.), benzene (100%), toluene (100%), benzyl alcohol (100%), benzyl benzoate (100%), benzyl ether (100%), methyl salicylate (100%), mono or dichloro-salicylate (100%), thiodiethanol (100%), formamide (100%), a fructose solution (about 100 mg/g to about 4 g/g aqueous solution, or 1 g/g solution), glycerol (about 0.1% to about 100% aqueous solutions, or 30% aqueous solution), chloral hydrate (about 1% to about 10% in water, or 10%), alcoholic glycerol (about 0.1% to about 99% alcoholic solutions, alcohols include methanol, ethanol, isopropanol, preferably ethanol, at 50% concentration), Visikol® (any grade or formula). In one embodiment, 30% aqueous glycerol was used as the preservation or storage solution.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Skeletal Visualization of Fetal Mice

### Visualization of Skeletons of Fetal Mice Specimens Stored in Isopropanol

Two fetal mouse specimens that had been eviscerated and stored in isopropanol 95%-100% (v/v) for up to one year, then placed into 50 mL 50 mM EDTA pH 7-8, for 60 min. The EDTA solution was then removed and replaced with 50 mL staining solution containing 0.03% Alizarin Red S. The staining solution was prepared fresh by dissolving 15 mg Alizarin Red S in 500 mL of 1% KOH (w/v). The specimens were stained for 22 hr, and then transferred to 50 mL DI H₂O for 2 hr. The DI H₂O was removed and 50 mL Visikol® ENDO™ was added for 4 hr at RT. After clearing, one specimen was stored in Visikol® ENDO™ and the other was stored in 70% (v/v) aqueous glycerol.

### Visualization of Skeletons of Fresh Fetal Mouse Specimens Stored in Glycerol

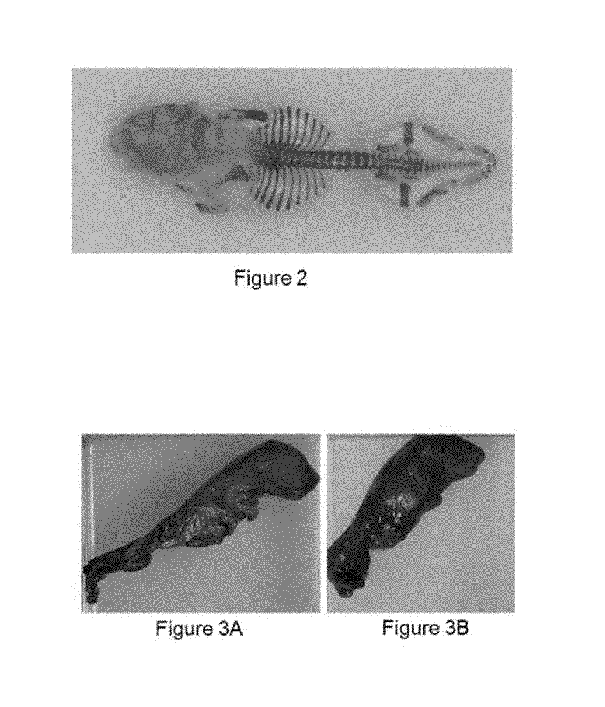

Two fetal mouse specimens were eviscerated and placed into 50 mL 50 mM EDTA, pH 7-8, for 60 min. The EDTA solution was removed and replaced with 50 mL staining solution containing Alizarin Red S, prepared as described in procedure A. The specimens were stained for 22 hr at RT, after which they were then transferred to 50 mL DI H₂O and incubated for 4 hr. The wash agent was then replaced with 50 mL 30% (v/v) glycerol clearing solution for 2 hr at RT. After clearing, the specimens were stored in 30% (v/v) aqueous glycerol (FIG. 2).

### Visualization of Skeletons of Fresh Fetal Mouse Specimens Stored in Visikol (Improved Clearing)

Two fetal mouse specimens were eviscerated and placed into 50 mL 50 mM EDTA and 1 M urea for 60 min (pH 7-8). This solution was removed and replaced with 50 mL staining solution containing Alizarin Red S, 1% KOH, prepared as described in procedure A. The specimens were stained for 24 hr, and then transferred to 50 mL DI H₂O and incubated for 3 hr. The DI H₂O was removed and replaced with 50 mL Visikol® ENDO™ for 2 hr at RT. After clearing, the specimens were stored in neat Visikol®.

### Visualization of Skeletons of Formalin-Fixed Fetal Mouse Specimens

Two fetal mouse specimens were eviscerated and placed into 50 mL 10% neutral buffered formalin. After 12 hours, the specimens were placed into 50 mM EDTA, 1 M urea, pH 7-8, for 60 min. This solution was removed and replaced with 50 mL staining solution containing Alizarin Red S, 1% KOH, prepared as described in procedure A. The specimens were stained for 24 hr, and then transferred to 50 mL DI H₂O and incubated for 3 hr. The DI H₂O was removed and replaced with 50 mL Visikol® ENDO™ for 2 hr at RT. After clearing, one specimen was stored in Visikol® ENDO™ and the other was stored in 70% (v/v) aqueous glycerol.

### Example 2

### Skeletal Visualization of Fetal Mice

Two fetal mouse specimens that have been eviscerated (without removing the skin) and stored in isopropanol 95%-100% (v/v) for up to one year, then placed into 50 mL 50 mM EDTA ph 7-8, for 60 mins. The EDTA solution was then removed and the specimens were washed with DI H₂O for 5 min, and then treated with 50 mL straining solution containing 0.03% Alizarin Red S. The straining solution was prepared fresh by dissolving 15 mg Alizarin Red S in 500 mL of 1% KOH (w/v). The specimens were strained for 22 hr, and then transferred to 50 mL Dl H₂O for 2 hr. The DI H₂O was removed and 50 Ml 30% (v/v) aqueous glycerol was added for 4 hr at RT. After clearing, one specimen was stored in Visikol® ENDO™ and the other was stored in 0% (v/v) aqueous glycerol.

### Example 3

### Skeletal Visualization of Adult Mice

Two adult mouse specimens were eviscerated and placed into 50 mL 50 mM EDTA, 1 M urea, pH 7-8, for 1 hr. This solution was removed and replaced with 50 mL staining solution containing Alizarin Red S, 1 M KOH, prepared as described in Example 1. The specimens were stained for 24 hr, after which they were then transferred to 50 mL DI H₂O and incubated for 3 hr. The DI H₂O was removed and 50 mL Visikol® ENDO™ was added for 2 hr at RT. After clearing, one specimen was stored in Visikol® ENDO™ and the other was stored in aqueous glycerol (70%, v/v).

### Example 4

### Skeletal Visualization of Fetal Rats

### Visualization of Skeletons of Fetal Rat Specimens Stored in Isopropanol

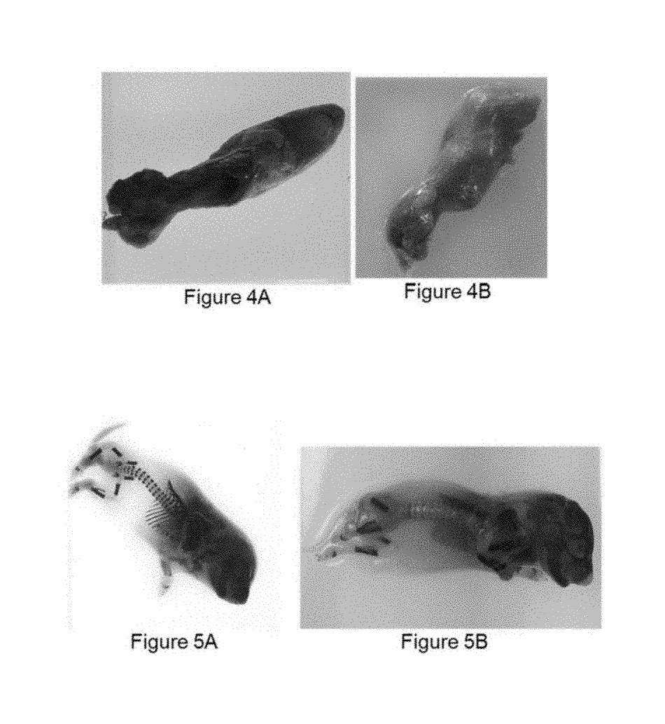

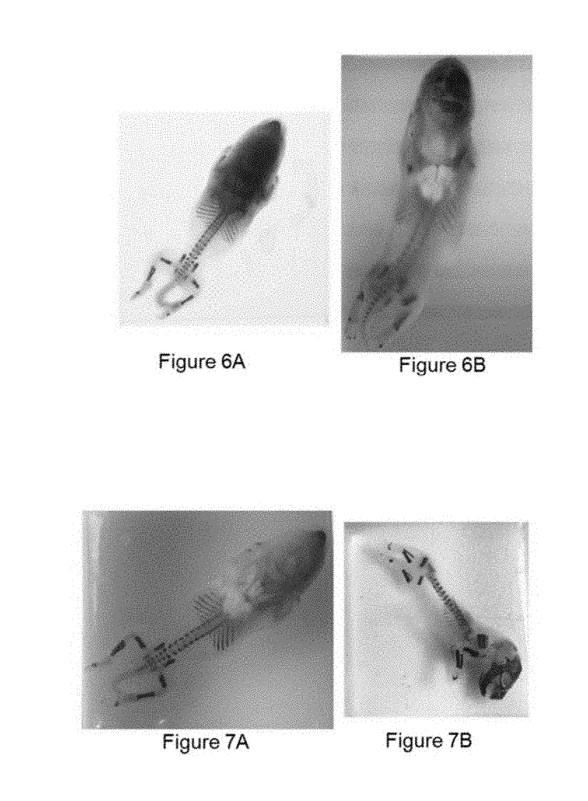

One fetal rat specimen was eviscerated and stored in isopropanol (95% v/v with water) up to 1 year (note: FIG. 2A is a dehydrated sample in 100% propanol, FIG. 2B is a previously-frozen sample). The fetal rat specimen was removed from the isopropanol and placed into 50 mL 100 mM EDTA, pH 7-8, for 2 hr (FIG. 3A a dehydrated specimen in EDTA, FIG. 3B a previously-frozen specimen in EDTA). The EDTA solution was removed and then replaced with 50 mL staining solution containing Alizarin Red S, 1% KOH, prepared as described in Example 1. The specimen was stained for 24 hr (FIG. 4A for a dehydrated specimen, FIG. 4B for a previously-frozen specimen), and then transferred to 50 mL DI H₂O and incubated for 6 hr (FIG. 5A for a dehydrated specimen, FIG. 5B for a previously-frozen specimen). The DI H₂O was then removed and cleared with 50 mL 30% glycerol, 0.05% Triton™ X-100 for overnight at RT (FIG. 6A for a dehydrated specimen, FIG. 6B for a previously-frozen specimen). After clearing, the specimen was stored in aqueous glycerol (30%, v/v).

### Example 5

### Skeletal Visualization of Fetal Rats

One fetal rat specimen was eviscerated (without removing the skin) and stored in isopropanol (95% v/v with water) up to 1 year. The fetal rat specimen was removed from the isopropanol and placed into 50 mL 100 mM EDTA, pH 7-8, for 2 hr. The EDTA solution was removed and the specimen was washed with DI H₂O for 5 min, and then treated with 50 mL straining solution containing Alizarin Red S, 1% KOH, prepared as described in Example 1. The specimen was stained for 24 hr, and then transferred to 50 mL DI H₂O and incubated for 6 hr. The DI H₂O was then removed and cleared with 50 mL 30% glycerol, 0.05% Triton X-100 for overnight at RT. After clearing, the specimen was stored in aqueous glycerol (30%, v/v).

### Visualization of Skeletons of Fresh Fetal Rat Specimens

One fetal rat specimen was eviscerated and placed into 50 mL 50 mM EDTA, 1 M urea, pH 7-8, for 3 hr. This solution was then removed and replaced with 50 mL staining solution containing Alizarin Red S, 1% KOH, prepared as described in Example 1. The specimen was stained for 48 hr, and then transferred to 50 mL DI H₂O and incubated for 4 hr, after which the DI H₂O was removed and replaced with 50 mL 30% glycerol (v/v), 0.05% Triton™ X-100 for overnight at RT. After clearing, the specimen was stored in 60% (v/v) aqueous glycerol.

### Visualization of Skeletons of Fresh Fetal Rat Specimens (Improved Wash Step)

One fetal rat specimen was eviscerated and placed into 50 mL of 50 mM EDTA solution containing 3 hr. The EDTA solution was removed and replaced with 50 mL staining solution containing Alizarin Red S, 1% (w/v) KOH, prepared as described in Example 1. The specimen was stained for 48 hr, and then transferred to 50 mL 1 M urea in DI H₂O and incubated for 4 hr. The urea solution was removed and replaced with 50 mL 30% glycerol for 4 hr at RT. After clearing, the specimen was stored in 30% (v/v) aqueous glycerol.

### Visualization of Skeletons and Cartilage of Stored Fetal Rat Specimens via Double Staining Method

One fetal rat specimen was eviscerated and stored in isopropanol (95% v/v with water) up to 1 year. The specimen was removed from the isopropanol and placed into 50 mL staining solution containing Alcian blue 8GX (75 mg in 500 mL of 1:4 acetic acid/ethanol, v/v) for 12 hr. The specimen was removed from the stain and placed into 95% (v/v) isopropanol. The specimen was removed from the isopropanol and placed into 50 mL 100 mM EDTA, pH 7-8, for 2 hr. The EDTA solution was removed and then replaced with 50 mL staining solution containing Alizarin Red S, 1% KOH, prepared as described in Example 1. The specimen was stained for 22 hr, and then transferred to 50 mL DI H₂O and incubated for 4 hrs. The DI H₂O was then removed and the specimen cleared with 50 mL 30% glycerol, 0.05% Triton™ X-100 for 4 hr at RT. After clearing, the specimen was stored in 40% (v/v with glycerol as diluent) Visikol® ENDO™.

### Example 6

### Skeletal Visualization of Fetal Rabbits

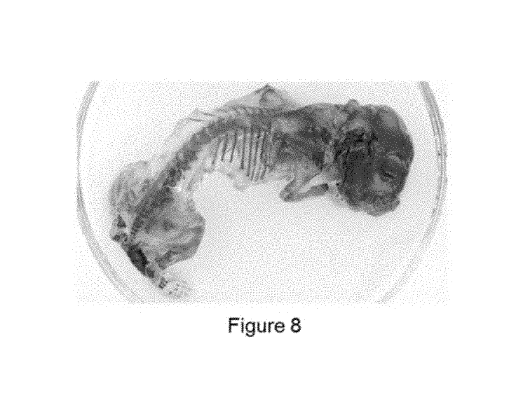

One fetal rabbit specimen was eviscerated and the fat pad between scapulae were removed along with the skin. The rabbit specimen was placed into 100 mL 100 mM EDTA, pH 7-8, for about 4 hr to about 8 hr. The EDTA solution was removed and the specimen was washed with DI H₂O for 1 hr, and then treated with 100 mL Alizarin Red S solution, prepared and described in Example 1. The specimen was stained for about 20 hr to 24 hr, and then transferred to 100 mL DI H₂O and incubated for at least 6 hr. The DI H₂O was then removed and replaced and 100 mL Visikol® ENDO™ solution for overnight at RT. After clearing, the specimen was stored in aqueous glycerol (40%, v/v, FIG. 8).

### Example 7

### Skeletal Visualization of Fetal Rabbits

One fetal rabbit specimen was eviscerated and the fat pad between scapulae were removed. The rabbit specimen was placed into 100 mL 100 mM EDTA, pH 7-8, for about 4 hr to about 8 hr. The EDTA solution was removed and replaced with 100 mL Alizarin Red S solution, prepared as described in Example 1. The specimen was stained for about 20 hr to 24 hr, and then transferred to 100 mL DI H₂O and incubated for at least 6 hr. The DI H₂O was then removed and replaced with 100 mL Visikol® ENDO™ solution for overnight at RT. After clearing, the specimen was stored in aqueous glycerol (40%, v/v, FIG. 8).

### Example 8

### Visualization of Calcium Oxalate Crystals in Arabidopsis thaliana

Leaves of mature Arabidopsis thaliana plant were removed from the plant. The leaves were placed into 10 mM EDTA solution for 1 hr. The EDTA solution was removed and replaced with 100 mL Alizarin Red S solution, prepared as described in Example 1. After 3 hr, the leaves were removed and placed into 10 mM urea. After 1 hr, the urea solution was then removed and cleared with 50 mL 30% glycerol, 0.05% Triton™ X-100 for 4 hr at RT. After clearing, the specimens were stored in 70% (v/v) aqueous glycerol.

### EQUIVALENTS

Those skilled in the art will recognize, or be able to ascertain, using no more than routine experimentation, numerous equivalents to the specific composition and procedures described herein. Such equivalents are considered to be within the scope of this disclosure, and are covered by the following claims.

1. A method of visualizing a calcified component in a tissue specimen, comprising:
a) contacting a tissue specimen with a chelating agent, the chelating agent binding to the calcified component;
b) staining the contacted tissue specimen with a calcium binding dye solution; and
c) clarifying the stained specimen with a clearing solution that makes visible the calcified components.

2. The method of claim 1, wherein the tissue specimen is a plant, fungi, vertebrate, embryo, adult, or a portion thereof.

3. The method of claim 1, wherein the tissue specimen is fresh, frozen, dehydrated and/or fixed with cross-linking, or precipitating.

4. The method of claim 1, wherein the chelating agent comprises ethylenediaminetetraacetic acid (EDTA), diethylenetriaminepentaacetic acid (DTPA), hydroxyethylethylenediamine triacetic acid (HEDTA), nitrilotriacetic acid (NTA), ethylene glycol tetraacetic acid (EGTA), or 1,2-bis(o-aminophenoxy)ethane-N,N,N′,N′-tetraacetic acid (BAPTA).

5. The method of claim 4, wherein the chelating agent comprises EDTA.

6. The method of claim 5, wherein the chelating agent comprises 0.1-100 mM EDTA.

7. The method of claim 1, wherein the dye comprises Alizarin Red, Alizarin Red S, Alcian Blue, or a combination thereof.

8. The method of claim 7, wherein the dye comprises Alizarin Red.

9. The method of claim 1, wherein the clearing solution comprises glycerol, water, and/or an aqueous surfactant mixture.

10. The method of claim 9, wherein the clearing solution comprises glycerol.

11. The method of claim 10, wherein the clearing solution comprises 15% to 70% glycerol in water.

12. The method of claim 1, wherein the staining step uses a double-stain technique.

13. The method of claim 1, wherein the specimen is washed before it is stained.

14. The method of claim 1, wherein the stained specimen is washed before it is clarified with the clearing solution.

15. A kit for visualizing a calcified component in a tissue specimen, comprising:
a chelating agent;
a calcium-binding dye; and
a clearing solution.

16. The kit of claim 15, wherein the chelating agent comprises ethylenediaminetetraacetic acid (EDTA), diethylenetriaminepentaacetic acid (DTPA), hydroxyethylethylenediamine triacetic acid (HEDTA), nitrilotriacetic acid (NTA), ethylene glycol tetraacetic acid (EGTA), 1,2bis(o-aminophenoxy)ethane-N,N,N′,N′-tetraacetic acid (BAPTA), or mixtures thereof.

17. The kit of claim 16, wherein the chelating agent is EDTA.

18. The kit of claim 15, wherein the dye comprises Alizarin Red, Alizarin Red S, Alcian Blue, or combinations thereof.

19. The kit of claim 18, wherein the dye comprises Alizarin Red.

20. The kit of claim 15, wherein the clearing solution comprises glycerol, water and/or an aqueous surfactant mixture.

21. The kit of claim 20, wherein the clearing solution comprises glycerol.

22. The kit of claim 15, further comprising dH₂O, KOH, a bacteriostatic, instructions, and combinations thereof.
